(19) United States
(12) Patent Application Publication
(10) Pub. No.: US 2024/0404679 A1
(43) Pub. Date: Dec. 5, 2024

(54) System for Performing Contextualized Neuroscience Assessments and Associated Methods

(71) Applicant: Immersion Neuroscience, Inc., Henderson, NV (US)

(72) Inventors: Jorge A. Barraza, Claremont, CA (US); Paul J. Zak, Loma Linda, CA (US)

(73) Assignee: Immersion Neuroscience, Inc., Henderson, NV (US)

(21) Appl. No.: 18/798,799

(22) Filed: Aug. 8, 2024

Related U.S. Application Data
(63) Continuation-in-part of application No. 17/974,609, filed on Oct. 27, 2022.
Application No. 63/531,944 is a continuation-in-part of application No. 17/874,114, filed on Jul. 26, 2022.
(60) Provisional application No. 63/531,944, filed on Aug. 10, 2023.
(60) Provisional application No. 63/272,304, filed on Oct. 27, 2021.
(60) Provisional application No. 63/227,544, filed on Jul. 30, 2021.

(51) Int. Cl. G16H 20/70

(52) U.S. Cl. CPC G16H 20/70

(57) ABSTRACT

Neurophysiologic assessment system includes an ingestion data hub for generating clean data from heart rhythm data, a neuroscience processing unit for generating primary metrics from the clean data, a contextual analysis unit for receiving and analyzing external contextual data, a behavior analysis unit for generating secondary metrics from the clean data, primary metrics, and external contextual data, and a workflow management unit for controlling the ingestion data hub, and neuroscience processing, contextual analysis, and behavior analysis units. Secondary metrics include at least one of emotional wellbeing, energy, mood, and peak immersion value for a given time frame. The external contextual data include at least one of location, proximity of related persons, time of day, day of a week, weather conditions, traffic conditions, measured activity levels, calendar events, biofeedback, survey results and information provided by the participant, tracked usage of software applications, engagement with social media, and sleep patterns.

### REFERENCE TO RELATED APPLICATIONS

The present application claims the benefit of U.S. Provisional Pat. App. No. 63/531,944, filed 2023 Aug. 10 and titled “System for Performing Contextualized Neuroscience Assessments and Associated Methods.” The present application is a Continuation-In-Part of U.S. patent application Ser. No. 17/874,114, filed 2022 Jul. 26 and titled “Immersion Assessment System and Associated Methods,” which claims priority to U.S. Provisional Pat. App. No. 63/227,544, filed 2021 Jul. 30 and titled “Immersion Assessment System and Associated Methods. The present application is also a Continuation-In-Part of U.S. patent application Ser. No. 17/974,609, filed 2022 Oct. 27 and titled “System for Simultaneously Assessing Psychological Safety in Real Time and Associated Methods,” which claims priority to U.S. Provisional Pat. App. No. 63/272,304, filed 2021 Oct. 27 and titled “System for Simultaneously Assessing Psychological Safety in Real Time and Associated Methods.” All of the above referenced applications are incorporated hereby in their entirety by reference.

### FIELD OF THE INVENTION

The present invention relates to the assessment of neuroscientific parameters. In particular, but not by way of limitation, the present invention relates to the assessment of neuroscientific parameters within context and, in some cases, in real time among two or more people.

### DESCRIPTION OF RELATED ART

Today, businesses make decisions based on what people “feel” or what they “like” using surveys, focus groups, or an executive's “intuition.” Science and history have shown that these decisions are only right roughly 17% of the time.

A variety of methods have been used to more rigorously measure people's engagement with a particular experience, such as an advertisement, media content, or live experience, and predicting participant behavior, although these methods have disadvantages in practical usage. Such methods include the following examples.
1) Eye-tracking: This method measures visual attention by using sensors to track the movement of a participant's eyes. However, the emotional impact or value of content on the participant cannot be measured. There is usually a high rate of data loss (e.g., 50% or more), especially with remote eye-tracking solutions, due to stringent lighting and head orientation requirements.
2) Automated facial coding: This method involves capturing a person's facial muscle movements using a camera while the participant is presented with media content, such as video clips on a computer. Popularized by the work of psychologist Paul Ekman, facial coding is one of the most widely utilized measures in neuromarketing as the data capture is simple and the data analysis can be automated using algorithms. However, academic research has shown that the facial coding method is poor at capturing emotions accurately (see, for example, L. F. Barrett, et al., “Emotional Expressions Reconsidered: Challenges to Inferring Emotion from Human Facial Movements,” Psychological Science in the Public Interest, vol. 20, Issue 1, pp. 1-68, Jul. 17, 2019 (https://journals.sagepub.com/doi/full/10.1177/1529100619832930 accessed Jul. 7, 2021)). Moreover, the technology is almost entirely focused on presenting content via a computer in a structured environment with sufficient lighting, where the participant is asked to remain still and keep their head in one position within a few feet of the camera.
3) Electroencephalogram (EEG): EEG devices use electrodes that are attached to specific locations on a participant's scalp to detect electrical activity in the participant's brain. There is a high variance in the quality of EEG devices. For instance, devices with only a few electrodes, while easy to use, are often unreliable and inaccurate in their readouts compared to medical-grade EEGs. On the other hand, medical grade EEG caps are cumbersome, uncomfortable to wear, and can only be used in a lab setting. Furthermore, correspondence between EEG data and specific emotions has not been solidly and scientifically established. The use of EEG devices is generally cost-prohibitive to scale for use in realistic experiences people have and is nearly impossible for multi-participant situations.
4) Galvanic Skin Response (GSR): GSR devices detect changes in sweat gland activity, which lead to changes in electrical properties of the skin measurable as, for instance, skin conductance. It is difficult to ensure accuracy and fidelity of GSR data, as they are highly dependent on the collection environment (e.g., variations in skin physiology, external temperatures in the 68 to 72 Fahrenheit range, etc.) and are incredibly sensitive to normal movement.
5) Implicit reaction time: Rooted in academic research on racial and gender biases, this analysis supposes that the reaction time (i.e., speed of response) of participants to specific stimuli is shortened when the brain is more strongly engaged in the activity. However, neither reliability nor predictive validity has been scientifically established for the correlation between implicit reaction time and real-world behaviors.

Further, knowing what people's brains value is imperative for creating a transformative experience. Rigorous neuroscience research in the past several decades has established a relationship between what a person is experiencing and the corresponding neurochemicals produced by that person's brain. In particular, the secretion of neurochemicals oxytocin and dopamine have been established as key signals showing that the brain values an experience. For instance, researchers have found connections between the presence of oxytocin and social behaviors such as trustworthiness, generosity, charitable giving (See, for example, 1) Zak, et al., “Oxytocin increases generosity in humans,” PLOS One 2 (11): e1128, 2007; 2) Zak, et al., “The neurobiology of trust,” Ann. N.Y. Acad. Sci 1032, pp. 224-227, 2004; 3) Barraza, et al., “Empathy toward strangers triggers oxytocin release and subsequent generosity,” Values, Empathy, and Fairness across Social Barriers, Ann. N.Y. Acad. Sci, 1167, pp. 182-189, 2009; 4) Barraza, et al., “Oxytocin infusion increases charitable donations regardless of monetary resources,” Hormones and Behavior, 60, pp. 148-151, 2011; 5) Lin, et al., “Oxytocin increases the influence of public service advertisements,” PLOS One 8 (2): e56934, 2013); 6) Alexander, et al., “Preliminary evidence of the neurophysiologic effects of online coupons: Changes in oxytocin, stress, and mood,” Psychology & Marketing, 32 (9), pp. 977-986, 2015; and 7) Zak, et al., “The neurobiology of collective action,” Frontiers in Neuroscience, 7 (211), pp. 1-9, 2013).

Physiologically, the presence of oxytocin has been shown to correspondingly modulate the heart's rhythms in measurable ways (See, for example, 1) Porges, “The polyvagal theory: phylogenetic substrates of a social nervous system,” International Journal of Psychophysiology, 42 (2001), pp. 123-146, 2001; 2) Thayer, et al., “Claude Bernard and the heart-brain connection: Further elaboration of a model of neurovisceral integration,” Neuroscience and Biobehavioral Reviews, 33 (2009), pp. 81-88, 2009; 3) Kemp, et al., “Oxytocin increases heart rate variability in humans at rest: Implications for social approach-related motivation and capacity for social engagement,” PLOS One 7 (8): 344014, 2012; 4) Norman, et al., “Oxytocin increases autonomic cardiac control: Moderation by loneliness,” Biological Psychology 86 (2011), pp. 174-180, 2011; 5) Barraza, et al., “The heart of the story: Peripheral physiology during narrative exposure predicts charitable giving,” Biological Psychology 105 (2015), pp. 138-143, 2015; 6) Jurek, et al., “The oxytocin receptor: From intracellular signaling to behavior,” Physiol. Rev., 98, pp. 1806-1908, 2018; and 7) Gutkowska, et al., “Oxytocin revisited: Its role in cardiovascular regulation,” Journal of Neuroendocrinology, 24, pp. 599-608, 2012). In addition, the binding of dopamine to the prefrontal cortex is associated with the release of adrenocorticotropic hormone (ACTH) in a person's blood stream, which in turn produces changes in the person's heart rhythm (Pivonello, R., Ferone, D., Lombardi, G., Colao, A., Lamberts, S. W., & Hofland, L. J. (2007). Novel insights in dopamine receptor physiology. European journal of endocrinology, 156 (1), S13.). Consequently, by monitoring subtle changes in heart rhythms, the brain's neurochemical response to an experience can be inferred such that heart rhythm data, such as collected using a photoplethysmogram (PPG), can be used to assess the person's reaction to an experience.

For instance, if a person is emotionally resonating with an experience, e.g., watching a movie or a commercial, sitting in a class, or working with a team, that person's brain typically releases oxytocin both into the brain and via the pituitary gland into the bloodstream. As oxytocin is simultaneously released into the brain and the bloodstream, a change in the oxytocin level in the blood reflects release of oxytocin in the brain. In the bloodstream, oxytocin binds to the vagus nerve and heart, thereby subtly changing the heart's rhythms (Norman et al., 2011, cited above). Thus, measurement of changes in heart rhythms can be used to infer the person's engagement with an experience at a particular moment in time.

An indicator of such a state of engagement is “immersion.” Immersion is defined as a biological state of attention and emotional resonance in the brain, as measurable by changes in the balance of neurochemicals in the brain and in the blood stream and their neuroelectrical analogs. Due to the effects of these changes on the peripheral nervous system, a person's level of immersion can also be inferred by monitoring subtle changes in the person's heart rhythms, as established in scientific research cited above. For instance, analysis of immersion has shown to predict what people will do and remember after an experience with over 80% accuracy.

Beyond immersion, the concept of psychological safety as an indicator of collaboration effectiveness has been extensively explored in the past 20+ years. Pioneered in the 1990s by Harvard University researcher Amy Edmondson, measurement of psychological safety in real life contexts is limited to self-reporting and surveys.

Unlike the psychological definition provided by Edmondson, psychological safety can be defined as a brain state that signals psychological comfort in social environments. High levels of psychological safety are considered to be an indication that a person feels relaxed, comfortable and ready to interact with others.

In the present disclosure, psychological safety is defined as a specific and measurable neurological state of readiness to engage in the social environment. This state encourages risk-taking, creativity, a sense of belonging, and a capacity to learn. Others have defined it as a subjective “psychological” belief that a person safe in a group and can take risks in a social environment (see, for example, Edmondson, “Psychological Safety and Learning Behavior in Work Teams,” Administrative Science Quarterly, Vol. 44, No. 2 (June 1999), pp. 350-383).

For instance, within a work environment, organizations are more likely to innovate quickly, unlock the benefits of diversity, and adapt well to change when employees feel comfortable asking for help, sharing suggestions with leadership, or challenging the status quo without fear of negative social consequences. Successfully creating an agile organizational structure that empowers teams to tackle problems quickly by operating outside of bureaucratic or siloed structures requires a strong degree of psychological safety. Research has shown that teams that possess two key aspects of their culture-interpersonal trust and psychological safety—are much more likely to be effective and reach performance objectives than are teams without these characteristics (Nowack, K. & Zak, P. J. (2021). Sustain high performance with psychological safety. American Society for Training & Development. February ISBN: 9781952157776).

Unlike engagement, which has been measured using a variety of in-lab and in situ measurement techniques, psychological safety analyses are mostly based on self-reported measurement mechanisms such as surveys. Therefore, existing assessment techniques for psychological safety are generally considered subjective and may be influenced by a variety of factors such as the wording of the survey questions, the timing of the survey administration, and environmental factors like concerns for their reputation if they were to respond to the survey in a negative manner.

The aforementioned and other existing methods only provide quantitative assessment of psychological safety, relying solely on self-reporting (e.g., surveys), failing to utilize neuroscientific approaches. Further, they are also disadvantageous for practical usage in terms of hardware costs, effort, expertise, sensitivity, and accuracy.

### SUMMARY OF THE INVENTION

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

In accordance with the embodiments described herein, there is described a neurologic immersion assessment system for assessing immersion levels of one or more experience participants based on heart rhythm data collected from the one or more people during an experience. The system includes an ingestion data hub for processing the heart rhythm data to provide clean data, and a neuroscience processing unit for analyzing the clean data and providing analysis results including primary metrics. The system further includes a behavior analysis unit for further analyzing the clean data and the analysis results to provide secondary metrics, and a workflow management unit for controlling the ingestion data hub, the neuroscience processing unit, and the behavior analysis unit. As used herein, clean data includes heart rhythm time series data that have been processed, for example, by removing illogical information (e.g., heart rate above or below specified thresholds), aligning incoming data from multiple experience participants with timing specific for a specific event, and/or calibrating the incoming data according to sensor type.

In accordance with a further embodiment, the immersion assessment system includes a content control unit, interfaced with at least the neuroscience processing unit, for presenting the experience to the one or more experience participants and correlating the analysis results with specific parameters and timing associated with the experience.

In accordance with the embodiments described herein, there is described a neurophysiologic system for assessing psychological safety levels of one or more experience participants based on heart rhythm data collected from the one or more people during a specified time frame. The system includes an ingestion data hub for processing the heart rhythm data to provide clean data, and a neuroscience processing unit for analyzing the clean data over a specific time period at predetermined intervals and providing analysis results including primary metrics. The system further includes a behavior analysis unit for further analyzing the clean data and the analysis results to provide secondary metrics, and a workflow management unit for controlling the ingestion data hub, the neuroscience processing unit, and the behavior analysis unit.

In accordance with a further embodiment, the neurophysiologic assessment system includes a content control unit, interfaced with at least the neuroscience processing unit, for presenting the experience to the one or more experience participants and correlating the analysis results with specific parameters and timing associated with the experience.

In accordance with an embodiment, a neurophysiologic assessment system for quantitatively assessing psychological safety levels of one or more experience participants within an experience and predicting participant behavior during and after the experience is described. The system includes an ingestion data hub for receiving heart rhythm data collected from the participant during the experience and processing the heart rhythm data to provide clean data. The system also includes a neuroscience processing unit for receiving and analyzing the clean data over a specific time period at predetermined intervals to generate primary metrics. The system further includes a behavior analysis unit for receiving and analyzing the clean data and the primary metrics to generate secondary metrics, and a workflow management unit for controlling the ingestion data hub, the neuroscience processing unit, and the behavior analysis unit.

In accordance with a further embodiment, associated methods of using the neurophysiologic assessment system herein described are also disclosed.

In accordance with another embodiment, a method for assessing psychological safety levels of one or more participants includes collecting heart rhythm data from each one of the one or more participants, cleaning the heart rhythm data to produce clean data, analyzing the clean data over a specific time period at predetermined intervals, and generating analysis results including primary metrics.

In accordance with a further embodiment, a method for quantitatively assessing psychological safety levels of a participant within an experience is disclosed. The method includes collecting heart rhythm data from the participant during the experience, cleaning the heart rhythm data to produce clean data, analyzing the clean data over a specific time period at predetermined intervals, and generating analysis results including primary metrics.

In another embodiment, the method further includes further analyzing the clean data and the analysis results to generate secondary metrics. In certain embodiments, the secondary metrics include at least one of an analysis of the primary metrics over a duration of the experience, an analysis of the primary metrics during a specified interval within the experience, and a comparison to a database of norms based on the primary metrics.

In a further embodiment, the method further includes receiving heart rhythm data from a plurality of participants, wherein the secondary metrics include an aggregated analysis of the primary metrics from the plurality of participants.

In certain embodiments, the neurophysiologic assessment system for quantitatively assessing immersion levels and/or psychological safety levels further includes a contextual analysis unit for integrating contextual information into the analysis, thus enabling the quantitative assessment of the emotional wellbeing over time of one or more participating individuals. In embodiments, the contextual information includes, and are not limited to, location, proximity of related persons, time of day, day of a week, weather conditions, traffic conditions, measured activity levels, calendar events, biofeedback, survey results and information provided by the participant, tracked usage of software applications, engagement with social media, and sleep patterns. and others.

In a further embodiment, a method for quantitatively assessing the emotional wellbeing over time of one or more participating individuals is disclosed. The method includes collecting heart rhythm data from the participating individual over the course of one hour or more, cleaning the heart rhythm data over time, analyzing the clean data in an ongoing manner at predetermined intervals, and generating analysis results including primary and secondary metrics. In certain embodiments, the method further includes collecting contextual information simultaneously or sequentially with the heart rhythm data, cleaning the heart rhythm over time may include correlating the contextual information with the heart rhythm data, and analyzing the clean data includes analyzing the heart rhythm data over time and in context.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF DRAWINGS

FIG. 1 shows a block diagram of a system for assessing the immersion level of one or more participants with presented content and experiences and predicting experience participant behavior, in accordance with an embodiment.

FIG. 2 is a block diagram of a computing system for assessing the immersion level of one or more participants, in accordance with an embodiment.

FIG. 3 shows a flow diagram for using a system for assessing the immersion level of one or more participants with presented content and experiences, in accordance with an embodiment.

FIG. 4 shows an exemplary graph of processed heart rhythm or cardiac data as measured, along with an illustration of exemplary steps in analyzing the data presented in the graph, in accordance with an embodiment.

FIG. 5 shows a flow diagram for performing the steps corresponding to the illustration in FIG. 4, in accordance with an embodiment.

FIG. 6 shows a flow diagram for using a system for assessing the psychological safety level of one or more participants with presented content and experiences, in accordance with an embodiment.

FIG. 7 shows a block diagram of a system for assessing and/or predicting emotional well-being of one or more individuals, in accordance with an embodiment.

FIG. 8 shows a flow diagram for using a system for assessing and/or predicting emotional well-being of one or more individuals, in accordance with an embodiment.

FIG. 9 is a table summarizing the results of machine learning estimations in classifying Mood based on Immersion, in accordance with embodiments.

FIG. 10 is a table summarizing the results of machine learning estimations using Energy as the dependent variable, in accordance with embodiments.

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents.

### DETAILED DESCRIPTION OF THE INVENTION

Knowing what people's brains value is imperative for creating a transformative experience and has led to the proliferation of methods for assessing engagement using surveys, tests, and biometric measurements such as discussed above. Today, companies use such methodologies, sometimes referred to as neuromarketing, consumer neuroscience, or applied neuroscience, in fields such as advertising, marketing, training, and entertainment.

Rigorous neuroscience research in the past several decades has established a relationship between what a person is experiencing and the corresponding neurochemicals produced by that person's brain. In particular, the secretion of neurochemicals oxytocin and dopamine have been established as key signals showing that the brain values an experience. For instance, researchers have found connections between the presence of oxytocin and social behaviors such as trustworthiness, generosity, charitable giving (See, for example, 1) Zak, Stanton, Ahmadi, 2007; 3) Zak, Kurzban, Matzner, 2005; 3) Barraza, Zak, 2009; 4) Barraza, Mccullough, Ahmadi, Zak, 2011; and 5) Lin, Gerwal, Morin, Johnson, Zak 2013), purchases (Alexander, Tripp & Zak, 2015) and collective action (Zak & Barraza 2013)).

Physiologically, the presence of oxytocin has been shown to correspondingly modulate the heart's rhythms in measurable ways (See, for example, 1) Porges, 2001; 2) Thayer, Lane, 2009; 3) Kemp, Quintana, et al., 2012; 4) Norman, Cacioppo, et al, 2011; 5) Barraza, Terris, et al., 2015; 6) Jurek, Neumann, 2018; 7) Gutkowska, Jakowski, 2012). As an example, the presence of oxytocin affects the level of adrenocorticotropic hormone (ACTH) in a person's blood stream, which in turn produces changes in the person's heart rhythm. Consequently, by monitoring subtle changes in heart rhythms, the brain's neurochemical response to an experience can be inferred such that heart rhythm data, such as collected using photoplethysmography (PPG), can be used to assess the person's reaction to an experience.

For instance, if a person is emotionally resonating with an experience, e.g., watching a movie or a commercial, sitting in a class, or working with a team, that person's brain typically releases oxytocin both into the brain and via the pituitary gland into the bloodstream. As oxytocin is simultaneously released into the brain and the bloodstream, a change in the oxytocin level in the blood generally reflects the activity of oxytocin in the brain. In the bloodstream, oxytocin binds to the vagus nerve and heart, thereby subtly changing the heart's rhythms (Norman et al., 2011). Thus, measurement of changes in heart rhythms can be used to infer the person's engagement with an experience at a particular moment in time.

An indicator of such a state of engagement is “immersion.” Immersion is defined as a biological state of attention and emotional resonance in the brain, measurable by changes in the balance of neurochemicals in the blood stream. Due to the effects of these neurochemical changes on the peripheral nervous system, a person's level of immersion can also be inferred by monitoring subtle changes in the person's heart rhythms, as established in scientific research cited above. For instance, analysis of immersion has been shown to predict what people will do and remember after an experience with over 80% accuracy.

In other words: 1) Immersion is a neurologic state of attention and emotional resonance with an experience; and 2) The state of immersion is predictive of experience outcomes. For instance, if immersion is high for an advertisement, that ad will be better remembered by a consumer and will predispose the consumer to take action (e.g., purchase, share on social media).

Due to recent advances in PPG sensors found in many common wearable devices, immersion levels can be assessed using commercial wearable devices as well as built-in smartphone cameras. Therefore, a system for simultaneously assessing immersion levels of multiple people using heart rhythm data via PPG sensing is described herein. That is, using PPG sensors that are widely available in smartwatches and fitness trackers, changes in patterns in heart rate, and the neurochemistry changes associated therewith, may be analyzed to simultaneously assess immersion levels of a large number of people outside of a laboratory environment. Included are also other sensing devices that enable obtaining heart rhythm data, such as built-in cameras on smartphones that utilize finger contact over the camera lens (see Coppetti, et al., 2017). The relevant heart rhythm data may include, for example, heart rate, heart rate variability, pulse rate variation, and other heart activity information.

As described herein, an immersion assessment system enables simultaneous heart rhythm data capture and assessment for one or more participants, along with a variety of interfaces (e.g., mobile, web, and desktop applications) to provide feedback to stakeholders for reporting and workflow management. For instance, the immersion assessment system of the present disclosure enables simultaneous evaluation of immersion levels of multiple participants' experience synchronously or asynchronously, thus providing accurate behavioral prediction.

It is noted that, within the present disclosure, the term “experience” may cover, for instance, pre-recorded media (such as entertainment content, training sessions, and educational videos), market research scenarios (e.g., staged settings with controlled variables like product experiences), and live events. That is, within the present disclosure, the participants in the various experiences encompass more than passive audiences, and experience participants may be actively engaged with presented scenarios, such as a mock shopping experience, a rock concert, or a live seminar.

In an embodiment, the immersion assessment system includes a distributed neuroscience software platform for collecting data from smartwatches or fitness sensors of multiple experience participants to directly measure, second by second, what an experience participant's brain values, enabling real-time, moment-by-moment, assessment of the experience participants' immersion levels, collected simultaneously from multiple experience participants. The assessments may be aggregated to provide additional insights in situations that would not be possible in a controlled, laboratory environment. That is, unlike previous engagement analysis systems that are limited to data collection from one or a few experience participants at a time within a confined setting such as an observation room or a laboratory, the immersion assessment system of the present disclosure enables near real-time collection and viewing of data from a plurality of viewers of specific media content, or even attendees of live events such as educational seminars, for nearly any type of experience in a non-obtrusive way using commonly-used PPG data collection wearables such as smartwatches and fitness trackers, or using PPG approaches using a built-in camera of a smart device, such as fingertip contact photoplethysmography (e.g., measuring finger pulse by contacting a fingertip to a built-in camera of a smart device) or non-contact photoplethysmography (e.g., using the built-in camera of a smart device to measure heart rhythm data). Heart rhythm measurement may be performed by approaches other than PPG, as long as the heart rhythm data can be collected with sufficient accuracy and resolution to enable performance of the analytic processes described below.

More particularly, the immersion assessment system of the present disclosure uses heart rhythm data to assess two key indicators of neurologic immersion, namely: 1) attention to the experience; and 2) emotional resonance. As a person's attention increases during an experience, the activity in the person's brain's prefrontal cortex causes an increase in sympathetic activity measurable from cardiac (or equivalently, heart rhythm) data. Also, as discussed above, emotional resonance is associated with the brain's synthesis of the neurochemical oxytocin, which increases activity of the vagus nerve, thus altering the person's heart rhythm in detectable ways. The immersion assessment system of the present disclosure quantifies the neurologic response to a given experience by measuring changes in the heart rhythm and analyzing the measurements for corresponding indication of brain activity. Taking real-time heart rhythm data from one or simultaneously from a plurality of individuals sharing an experience, then processing the data using measured changes in heart rhythms, the immersion assessment system of the present disclosure enables simultaneous assessment of the immersion level of a plurality of experience participants essentially in real-time.

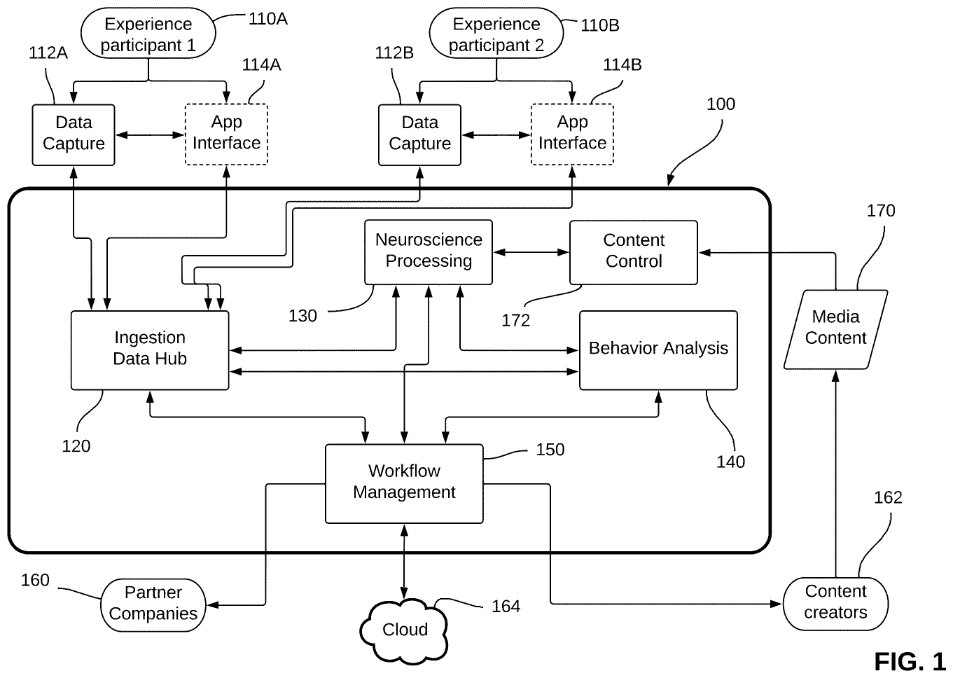

Turning now to the figures, FIG. 1 shows a block diagram of a system for assessing the level of immersion with an experience and predicting behavior of one or more experience participants, in accordance with an embodiment. As shown in FIG. 1, an immersion assessment system 100 interfaces with one or more experience participants (shown as 110A and 110B) through a data capture mechanism 112A and 112B, respectively. Experience participants 110A and B may be, for example, test participants being shown a film clip, a participant at a seminar, a movie goer, or an event attendee. It is noted that, while only two bubbles representing experience participants 110A and 110B are shown in FIG. 1, data capture may be simultaneously performed for just one participant, two or more experience participants, who may be simultaneously involved in the same experience, in the same experience at staggered times, or in different experiences at the same time.

Data capture mechanism 112A and 112B may be a device capable of capturing real-time heart rhythm data of the respective experience participant. As an example, data capture mechanism is a smartwatch or a fitness tracker worn by the experience participant to capture real-time heart rhythm data of experience participant. Alternatively, the experience participant may be directed to use the PPG capture feature of a smart device (e.g., the camera of a smart phone) while participating in the experience. While only two experience participants 110A and 110B are shown in FIG. 1, immersion assessment system 100 may be interfaced with just one experience participant or a plurality of experience participant members, with each experience participant associated with their own data capture mechanism (e.g., a smartwatch or fitness tracker worn by that experience participant). The heart rhythm data of the one or more experience participants may be transmitted to immersion assessment system 100 via a wired or wireless (e.g., Bluetooth® connection or other) connectivity mechanism in real-time or some time post experience.

Optionally, each experience participant may interact with an application interface (e.g., 114A and 114B as shown in FIG. 1) on a mobile device or a computer. Application interface may include, for example, a mobile application configured for communicating with immersion assessment system 100 and providing an interactive user interface for each experience participant. For instance, the application interface may display the experience to be assessed (e.g., media content, advertisement, event recording, or live event), provide an interface for each experience participant to adjust user settings, monitor the data capture mechanism, and/or send and receive information from immersion analysis system 100. Further, immersion analysis system 100 may be configured for accommodating a variety of data capture mechanisms and application interfaces (e.g., a Fitbit® fitness tracker connected via an iOS® operation system application as well as a Garmin® fitness tracker connected via an Android® operating system).

Continuing to refer to FIG. 1, immersion analysis system 100 includes an ingestion data hub 120 for interfacing with the experience participant(s) via data capture mechanism and/or application interface. Ingestion data hub 120 performs a variety of tasks such as pairing data from a specific experience participant with a specific event to be analyzed, cleaning the incoming heart rhythm data to remove illogical information (e.g., heart rate above or below specified thresholds), aligning incoming data from multiple experience participants with timing specific for a specific event, and calibrating the incoming data according to sensor type. Ingestion data hub 120 thus receives and processes the incoming heart rhythm data from one or more experience participants to provide clean data.

Immersion analysis system 100 also includes a neuroscience processing unit 130. Neuroscience processing unit 130 analyzes the clean data from ingestion data hub 120 to generate analytical results, such as primary metrics such as immersion and psychological safety by correlating received heart rhythm data with established neurochemical analyses, such as described above. Neuroscience processing unit 130 may also perform analyses such as the identification of key moments within the experience being analyzed, and the grouping of the experience timeline into time periods of high or low immersion. As an example, the grouping of the experience timeline into time periods of high or low immersion may be performed using a process referred to herein as “pilling,” as will be described in further detail below.

In the exemplary embodiment shown in FIG. 1, the immersion assessment system 100 further includes a behavior analysis unit 140. Behavior analysis unit 140 may receive the clean data from ingestion data hub 120 and the analysis results from neuroscience processing unit 130 to perform further analyses such as, for instance, aggregate profiling, vertical analysis, and pattern analysis. Optionally, neuroscience processing unit 130 and/or behavior analysis unit 140 may perform additional functions such as the calculation of secondary metrics (e.g., comparison of the primary metrics with established norms), identifying and clipping key moments in the experience, and generating summary reports (e.g., norm comparison, key moments (high and low points), participant breakdown, correlating key moments with specific points in the experience agenda, generating annotated video of the experience with immersion assessment results). The primary and/or secondary metrics may optionally be sent via ingestion data hub 120 to be displayed to experience participant 110 via, for example, application interface 114.

Immersion assessment system 100 of FIG. 1 further includes a workflow management unit 150. Workflow management unit 150 may include, for example, interfaces with ingestion data hub 120, neuroscience processing unit 130, and/or behavior analysis unit 140 for receiving and aggregating data from each of these system components. Workflow management unit 150 may also provide an interface between immersion assessment system 100 with external stakeholders, such as partner companies 160, who are users or clients of the immersion assessment system 100 or content creators 162, or provide aggregated data or analysis history to a cloud server 164. As an example, content creators 162 may include companies or personnel who produce the experience (e.g., event or media content 170) being assessed by the immersion assessment system 100. As another example, content creators may include content (or experience) participants who are managing the content/experience using the immersion assessment system 100 to organize the content/experience, invite selected experience participants to participate, and execute the measurement. Workflow management unit 150 may include a website or user interface for displaying, for instance, details related to experience participants 110 and media content 170, creation and management of experiences to be assessed, as well as data and analysis results visualization in real-time during the experience and/or after the conclusion of the experience. It is noted that media content 170 may be, for instance, a video recording of a live experience, or pre-recorded content presented to one or more experience participants.

In an example, media content 170 is provided by content creators 162 to a content control unit 172 for use in presenting the experience to be assessed (e.g., audiovisual content or online event) to experience participant 110 and in correlating the analysis results of neuroscience processing unit 130 with specific event timing of media content 170. Furthermore, content control unit 172 may provide media management functions to enable secure streaming of media content 170 to specific experience participants 110, or even adjust the content provided to each experience participant 110 according to the real-time analysis results from neuroscience processing unit 130.

It is noted that, while content control unit 172 is shown in FIG. 1 as being interfaced with neuroscience processing unit 130, content control unit 172 may be additionally or alternatively interfaced with ingestion data hub 120, behavior analysis unit 140, and/or workflow management unit 150. It is further noted that, while ingestion data hub 120, neuroscience processing 130, behavior analysis unit 140, workflow management unit 150, and content control unit 172 are shown as distinct components within the immersion assessment system 100, two or more of these components may be combined in a single unit.

It is further noted that, immersion assessment system 100 may be contained in a specialized hardware system integrating the various components therein, or implemented within a standalone computing system, including a processor and memory with programming executable by the processor to perform the functions of ingestion data hub 120, neuroscience processing unit 130, behavior analysis unit 140, workflow management unit 150, and content control unit 172. Alternatively, certain aspects of ingestion data hub 120, neuroscience processing unit 130, behavior analysis unit 140, workflow management unit 150, and/or content control unit 172 may be performed by dedicated hardware or within cloud 164. For instance, by providing certain aspects of the components within immersion assessment system 100 within cloud 164, specific functionalities of immersion assessment system 100 may be provided in a Software-as-a-Service configuration.

The immersion assessment system of the present disclosure differs from existing engagement assessment systems in at least the following ways:
1. Immediate, real-time results. Existing neuroscience hardware is expensive and complicated to use, and requires controlled environments during use. Consequently, both the data collection and analysis of data can be slow, at times taking weeks to get results. The immersion assessment system of the present disclosure enables second-by-second data capture that is processed in real-time, such that the analysis results may be obtained during and immediately after a given experience.
2. Measure reactions in any environment. While neuroscience laboratory settings enable strict control of the environment, labs are not where people live. In contrast, the immersion assessment system of the present disclosure enables data collection and analysis in situ, wherever the experience is taking place.
3. Outcome focused. The immersion assessment system of the present disclosure analyzes the heart rhythm data collected and aggregates the results in a way that provides actionable information supported by proven scientific research.
4. Remote data management. The immersion assessment system of the present disclosure enables remote collection of the necessary input data (i.e., heart rhythm) using wearable sensors connected to the immersion assessment system via, for example, wireless, cellular, or Bluetooth technology. Thus, any experience to be assessed may be monitored remotely.
5. Content control. Optionally, the immersion assessment system of the present disclosure enables real time control of the experience to be presented to participants as, for example, a live experience. For instance, based on real time assessment of the immersion level of participants, the content may be modified or different content be presented to participants.
6. Anonymity. The data collection (i.e., heart rhythm data input) may be aggregated and anonymized such that the participants can feel comfortable during assessments. That is, as participants are able to interact with neuroscience collection and neuroscience analytics using typical software-as-a-service (SaaS) privacy gates and levels of anonymity. Thus, partner companies and content creators may find it easier to recruit and encourage participation by a larger number of experience participants, and comply with consumer privacy and protection laws.

Furthermore, the immersion assessment system of the present disclosure may enable additional features such as online event management (including sending of event invitations, attendee registration, and event start/stop) and display of real-time immersion level to participants and/or stakeholders. The analysis results may be viewed in terms of, for example, a second-by-second line chart showing fluctuations in immersion levels throughout the experience, benchmarked metrics comparing the analysis results for a specific experience to existing norms, and secondary metrics, such as psychological safety, average experience scores, length and depth of deep immersion or emotional disconnect, and demographics of individuals most likely to engage with a particular experience.

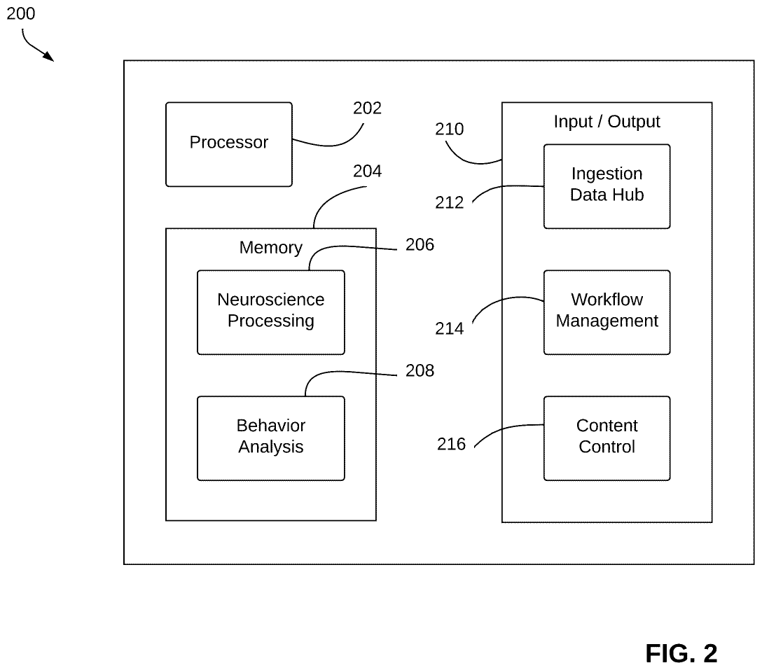

FIG. 2 is a block diagram of a computing system 200 for assessing the immersion level of one or more experience participants with presented content, in accordance with an embodiment. Computing system 200 may be a standalone computing system, in an example. Computing system 200 includes a processor 202 for controlling the operations of a memory 204, which includes programming such as neuroscience processing 206 (e.g., functions performed by neuroscience processing unit 130 of FIG. 1) and behavior analysis 208 (e.g., functions performed by behavior analysis unit 140 of FIG. 1), such programming being executable by processor 202. Processor 202 further controls an input/output interface 210, which is configured for receiving and transmitting data, such as heart rhythm data from one or more experience participants (e.g., experience participant 110 of FIG. 1), media content from content creators (e.g., media content 170 from content creators 162 of FIG. 1, and aggregated data and/or analysis history. In an example, input/output interface 210 includes an ingestion data hub 210, a workflow management unit 214, and a content control 216, which are analogous to ingestion data hub 120, workflow management unit 150, and content control unit 172 of FIG. 1. Thus, input/output interface may receive participant data and provide the received data to memory 204 to generate immersion analysis results, which then may be communicated outside of computing system 200.

As an alternative, portions of neuroscience processing, behavior analysis, data ingestion, workflow management, and/or content control may be performed outside of computing system 200, such as in the cloud (e.g., cloud 164 of FIG. 1), with input/output interface 210 controlling the flow of data between computing system 200 and the outside world.

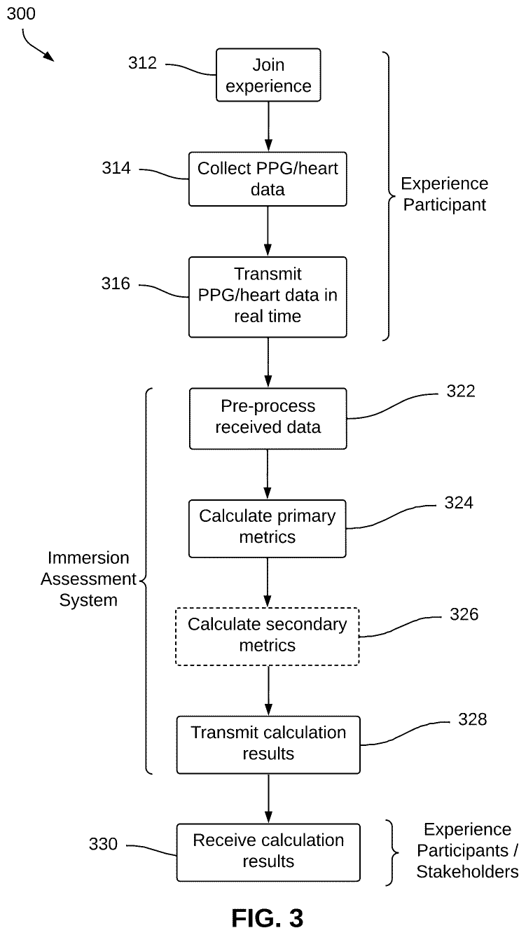

FIG. 3 shows a flow diagram for using a system for assessing the immersion level of an experience participant with presented content, in accordance with an embodiment. As shown in FIG. 3, in conjunction with FIG. 1, a process 300 begins when a participant (e.g., experience participant 110) opts to join an experience to be assessed (e.g., a live event or media content 170) in a step 312. Step 312 may include, for example, an opt-in agreement through an application interface or a manual signing of a waiver, in which the experience participant agrees to share physiological data (e.g., heart rhythm data) and in some cases demographic information collected during the experience. Then, in a step 314, heart rhythm data is collected from the participant, and the collected information is transmitted to the immersion assessment system (e.g., immersion assessment system 100). As shown in FIG. 3, steps 312, 314, and 316 are performed at devices controlled by the participant. It is noted that multiple participants may be performing steps 312, 314, and 316 at the same time to provide input data to the immersion assessment system.

Continuing to refer to FIG. 3, the heart rhythm data via PPG is received at the immersion assessment system to pre-process the received data in a step 322. Step 322 may be performed, for example, by ingestion data hub 120. Then primary metrics, such as immersion levels and psychological safety, are calculated in a step 324. Step 324 may be performed, for example, by neuroscience processing unit 130 by correlating the input data from participants with known neurophysiologic indicators, as described above. Optionally, secondary metrics may be calculated in a step 326 by, for example, behavior analysis unit 140 of FIG. 1. Such secondary metrics may include aggregated analyses of data from multiple participants and/or over the presentation time of the experience. The calculation results of the primary and/or secondary metrics are then transmitted to participants or stakeholders in a step 328, such as via application interface 114 and/or user interface aspects of workflow management unit 150 of the immersion assessment system 100. Finally, the calculation received by participants or other stakeholders (e.g., partner companies 160, content creators 162, or data aggregator in cloud server 164) in a step 330.

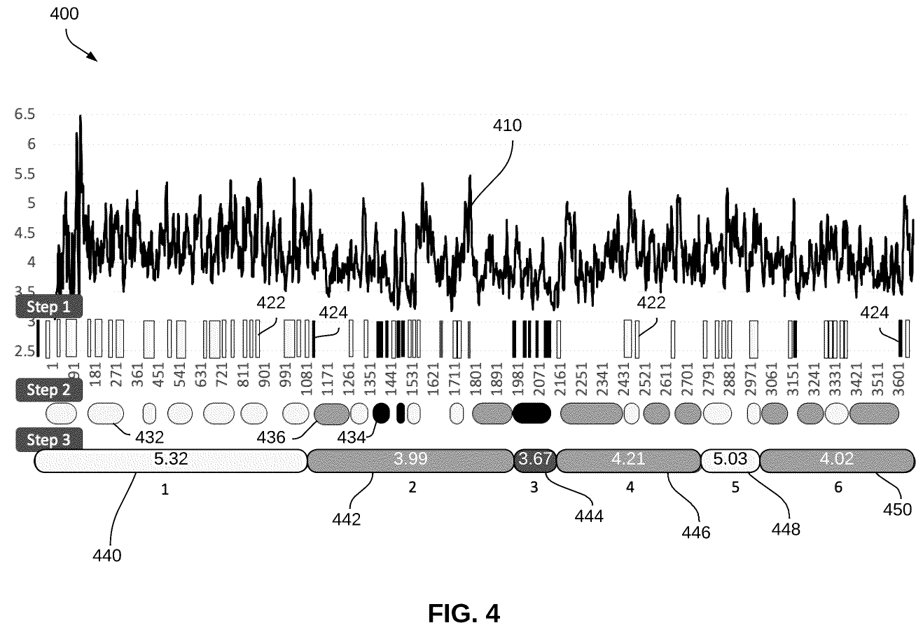

An example of data analysis that may be performed by immersion assessment system 100 is a clustering process to form what will be referred to as “pills” or clusters of time that segment an experience based on immersion scores. FIG. 4 shows an exemplary graph of processed heart rhythm data, along with an illustration of exemplary steps in analyzing the data presented in the graph, in accordance with an embodiment.

As described above, the immersion assessment system 100 receives heart rhythm data information via data capture mechanism 112 as raw data. This heart rhythm data information is processed by the various components within the immersion assessment system 100 such as ingestion data hub 120 and neuroscience processing unit 130 to provide a data graph 410 with the Y axis corresponding to calculated immersion value (normalized units) and the X axis corresponding to time (in seconds). That is, data graph 410 corresponds to the heart rhythm data that has been processed using established correlation between heart rhythms and measurable neurochemistry metrics (e.g., as discussed in the journal articles cited above) to provide a second-by-second immersion score.

Then, in a step 1, meaningful moments such as high immersion periods 422 (shown as light colored boxes), corresponding to peaks in the processed data graph 410, are identified. Similarly, low immersion periods 424 (shown as dark colored boxes), corresponding to low points in data graph 410, are identified.

In a step 2, trends in high immersion periods 422 and low immersion periods 424 are grouped into light “pills” (i.e., ovals) 432 and black pills 434. These groupings may be performed, for example, using thresholds involving the proximity of the identified high and low immersion periods, as well as the duration of each one of the high and low immersion periods. Additionally, neutral immersion periods (shown as gray pills), during which the experience participant is neither highly immersed or has low immersion are identified in lulls between the light or black pills.

Then, in a step 3, trends in the high, neutral, and low immersion periods are identified by grouping together adjacent pills. For instance, as shown in FIG. 4, a first time period 440 corresponds to relatively high immersion level by a participant being measured. Then the participant was neutral during a second time period 442, lost interest in a third time period 444, returned to neutral in a fourth time period 446, became highly immersed again in a fifth time period 448, then returned to neutral immersion level in a sixth time period 450. A numerical score (as shown within each time period 440, 442, 444, 446, 448, and 450) may be calculated for each of these time periods by averaging the calculated immersion levels within the total time encompassed by that time period.

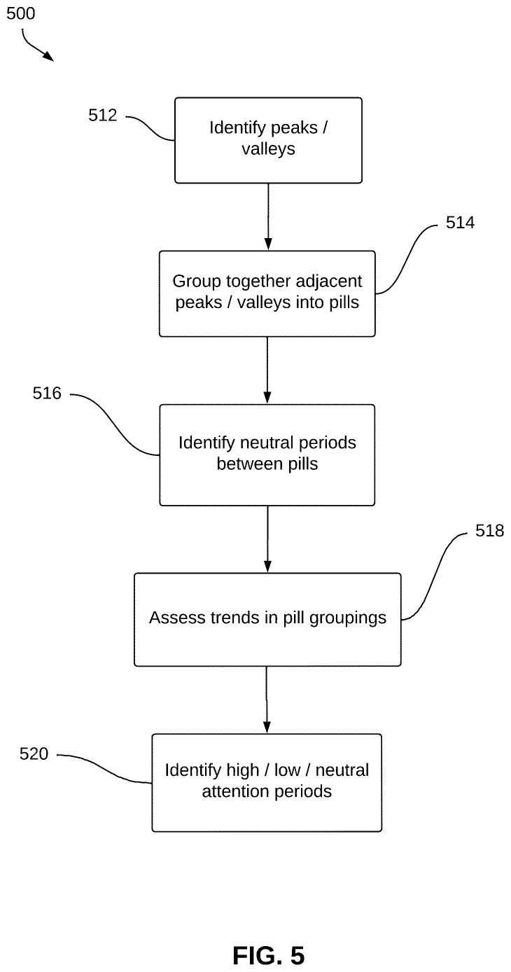

FIG. 5 shows a flow diagram for performing the steps corresponding to the illustration in FIG. 4, in accordance with an embodiment. As discussed relative to FIG. 4, a process 500 begins with a step 512 to identify the peaks and valleys in immersion, shown as the graph in FIG. 4. Adjacent peaks and valleys in immersion are grouped together into “pills” in a step 514. Further, neutral immersion periods between pills are identified in a step 516. Then, trends in the high/low/neutral pill groupings are assessed in a step 518, thus allowing identification of high/low/neutral immersion periods in a step 520.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel insights and advantages of this invention.

Accordingly, many different embodiments stem from the above description and the drawings. It will be understood that it would be unduly repetitious and obfuscating to literally describe and illustrate every combination and subcombination of these embodiments. As such, the present specification, including the drawings, shall be construed to constitute a complete written description of all combinations and subcombinations of the embodiments described herein, and of the manner and process of making and using them, and shall support claims to any such combination or subcombination.

As an example, while the described embodiments involve the use of PPG sensors to gather the data used in the immersion analysis, other types of heart activity sensors, such as electrocardiogra (ECG) sensors, may be used. While commonly used PPG sensors in smartwatches and fitness monitors are certainly convenient for simultaneously gathering heart rhythm data from multiple participants at live events or group settings, participants in certain types of more passive experiences (e.g., movie or television viewing) may be monitored using more static means, such as ECG sensors that require each participant to be provided with multiple electrodes, or utilizing the built-in camera of smartphones that require each participant to press and hold a finger onto the surface of the camera lens.

Further, while aggregation of data from multiple participants in real time provide heretofore unavailable analytic capabilities related to immersion, certain embodiments described herein may be useful in situations involving one or a few participants.

Neuroscientists have long understood the role of oxytocin, along with the heart-brain connection, in promoting positive social behavior. Humans evolved biological mechanisms to allow risk in social settings to happen, in other words, to gauge when a social environment is safe to take risks. For example, Porges, et al. (see Porges, et al., 2001, cited above) and Thayer, et al. (Thayer, et al., 2009, cited above) have clarified the connection between the heart and the brain, specifically identifying the role of oxytocin and vagal tone (e.g., heart rate variability (HRV)) in regulating social safety. Oxytocin is associated with increased cardiac vagal tone, namely the activity of the vagus nerve influence heart rhythms (See, for example, Kemp et al., “Depression, Comorbid Anxiety Disorders, and Heart Rate Variability in Physically Healthy, Unmedicated Patients: Implications for Cardiovascular Risk,” PLOS ONE, 7 (2) (2012); Norman et al., 2011, cited above), which is closely linked to the prefrontal-subcortical neural mechanism of self-regulatory function (Friedman, “An autonomic flexibility-neurovisceral integration model of anxiety and cardiac vagal tone,” Biol. Psychol., 74 (2), pp. 185-199, 2007; Park, et al., “From the heart to the mind: cardiac vagal tone modulates top-down and bottom-up visual perception and attention to emotional stimuli,” Frontiers in Psychology, 5 (278), 2014; Thayer, et al., 2009, cited above). According to the neuro-visceral integration model, cardiac vagal tone can index the functional integrity of the prefrontal-subcortical circuits (Friedman, 2007, cited above; Park, et al., 2014, cited above; Thayer, et al., 2009, cited above). Robust regulation of the heart via the vagus nerve, which can be indexed by higher resting HRV, is associated with more adaptive patterns of emotional responding and self-regulatory functioning, experiences of positive emotion, and resiliency to stress (see, for example, Friedman, 2007, cited above; Park, et al., 2014, cited above; Thayer, et al., 2009, cited above; Fabes, et al., “Regulatory control and adults' stress-related responses to daily life events,” Journal of Personality and Social Psychology, 73 (5), pp. 1107-1117, 1997; DiPietro, et al., “Reactivity and developmental competence in preterm and full-term infants,” Developmental Psychology, 28 (5), pp. 831-841, 1992; Oveis, et al., “Resting respiratory sinus arrythmia is associated with tonic positive emotionality,” Emotion, 9 (2), 2009). The relationship may be bi-directional, with an increase in positive emotions leading to greater resting HRV (Kok, et al., “How positive emotions build physical health: Perceived positive social connections account for the upward spiral between positive emotions and vagal tone,” Psychological Science, 24, pp. 1123-1132, 2013). As such, cardiac vagal activity plays an important role in the assessment of the brain state of psychological safety as experienced by an individual.

As discussed above, psychological safety, like immersion, is affected by the neurochemical oxytocin, which can facilitate prosocial behavior even among strangers. Further, research has demonstrated the causal effect of oxytocin on trust by infusing synthetic oxytocin, showing that those given synthetic oxytocin were twice as likely to show maximal trust in experimental scenarios (Kosfeld, M., Heinrichs, M., Zak, P. J., Fischbacher, U., & Fehr, E. (2005). Oxytocin increases trust in humans. Nature, 435 (7042), 673-676.). For instance, brain imaging experiments have shown that infusing people with oxytocin results in a marked reduction in fear-associated, brain-activity-enhancing psychological safety. That is, the more oxytocin your brain makes, the more you feel empathy toward others, connecting you emotionally and nudging you to invest in helping them.

In other words, the presence of oxytocin signals that a person is psychologically safe to be in a particular environment by reducing the natural wariness in a particular situation. While perceptions of capability, consistency, caring, candor, and authenticity as well as inherent factors such as culture, neurochemicals, and genetics all contribute to measures of psychological safety, measurement of the presence of oxytocin (either directly or through a secondary measurement of the physiological effects of oxytocin on the body), the level of psychological safety experienced by a person can be quantified.

Co-pending U.S. Provisional Patent Application Ser. No. 63/227,544, filed Jul. 30, 2021, entitled “Immersion Assessment System and Associated Methods” and incorporated herein in its entirety by reference, describes a system and associated methods for assessing immersion, which is an indicator of a participant's engagement with a particular experience. In particular, the immersion assessment system described in the aforementioned provisional application enables simultaneous PPG data capture and assessment for one or more individuals, along with a variety of interfaces (e.g., mobile, web, and desktop applications) to provide feedback to stakeholders for reporting and workflow management. Included are also other sensing devices that enable obtaining heart rhythm data, such as built-in cameras on smartphones that utilize finger contact over the camera lens (see Coppetti, Brauchten, Muggler, et al., (2017). Accuracy of smartphone apps for heart rate measurement. European Journal of Preventive Cardiology. 24 (12), 1287-1293). The relevant heart rhythm data may include, for example, heart rate, heart rate variability, pulse rate variation, and other heart activity information. For instance, the immersion assessment system enables simultaneous evaluation of immersion levels of multiple participants experience synchronously or asynchronously, thus providing accurate behavioral prediction, especially by comparing the assessment results to a database of norms based on the primary metrics. This database of norms may be, for example, created from an aggregated set of data collected experimentally from a variety of individuals across several studies (i.e., discrete observations with associated outcomes) using methods such as observation, surveys, and other assessments as prediction outcomes. In an embodiment, a database of norms may be integrated into the analytical algorithm implemented by neuroscience processing unit 130. As an example, the assessment scores may be weighted and normalized to fall within a numerical range of 1-100 according to a comparison to a database of norms, which database has been created from several behavioral studies of prediction outcomes.

It is recognized herein that, by analyzing heart rhythm data (such as PPG data) over a specific time period at predetermined intervals, psychological safety levels may be quantified without the use of subjective mechanisms such as surveys. That is, psychological safety may be assessed as a specific and measurable neurological state of readiness.

An exemplary process, in accordance with an embodiment, may include the following steps:
1. An experience participant is equipped with a heart data capture device, such as and not limited to a PPG-enabled smartwatch or fitness device.
2. The device outputs cardiac data, such as pulse, that is converted to heart rate data.
3. This heart rate data is delivered to the psychological safety assessment system, and is analyzed for changes in heart rate rhythms, along with other cardiac patterns associated with oxytocin release in the brain and binding on the vagus nerve.
4. The heart rate data is collected for a predetermined time period (e.g., 2 minutes or more) to observe sufficient variability in cardiac activity to determine the psychological safety indicator.
5. The system corrects the signal based on individual physiology and potential artifact or noise (e.g., movement, acceleration, or any other factor not typically associated with neurological sources of variability).
6. The system normalizes and derives a score of psychological safety.
7. Optionally, the psychological safety score is displayed for the user.

Alternatively, system 100 of FIG. 1 may be utilized as a system for assessing the level of psychological safety of one or more experience participants, or simply “participants,” in accordance with an embodiment. For instance, system 100 may be configured to provide an assessment of psychological safety may be utilized in the field of human wellness, which may be expanded outside of discrete events into occurrences in everyday life.

Application interface 114A may also display, for example, immersion scores for first experience participant 110A, as discussed in the aforementioned co-pending U.S. provisional patent application 63/227,544. Similar functionality may be provided to second experience participant 110B via an application interface 114B, which may be the same or different (e.g., different modality or operating system) compared to application interface 114A used by first experience participant 110A.

Neuroscience processing unit 130 in the context of psychological safety assessment may analyze the clean data from ingestion data hub 120 to generate analysis results as primary metrics, such as calculated psychological safety and immersion levels, by correlating received heart rhythm data with established neurochemical analyses, such as described above. Specifically, the data is processed to identify variation in the heart rhythm associated with both HRV in the high frequency range, as well as other heart rhythm patterns associated with oxytocin release in the brain. This process may occur in real time, requiring at least 2 minutes of data to begin outputting psychological safety scores for an individual or group. Neuroscience processing unit 130 presents the psychological safety scores along the experience timeline broken up into uniform time periods (e.g., 2-minute segments). Neuroscience processing unit 130 and/or behavior analysis unit 140 may perform additional functions such as the calculation of secondary metrics and generating summary reports as described above, participant breakdown (e.g., classification into categories ranging from very low psychological safety to very high psychological safety) and generating annotated video of the experience with psychological safety assessment results.

The psychological safety assessment system of the present disclosure differs from existing engagement assessment systems in that the system provides outputs based on quantitative data, namely the analysis of subtle changes in heart rhythms at predetermined intervals (e.g., every two minutes) over a specified time frame (e.g., during the first five minutes of a business meeting).

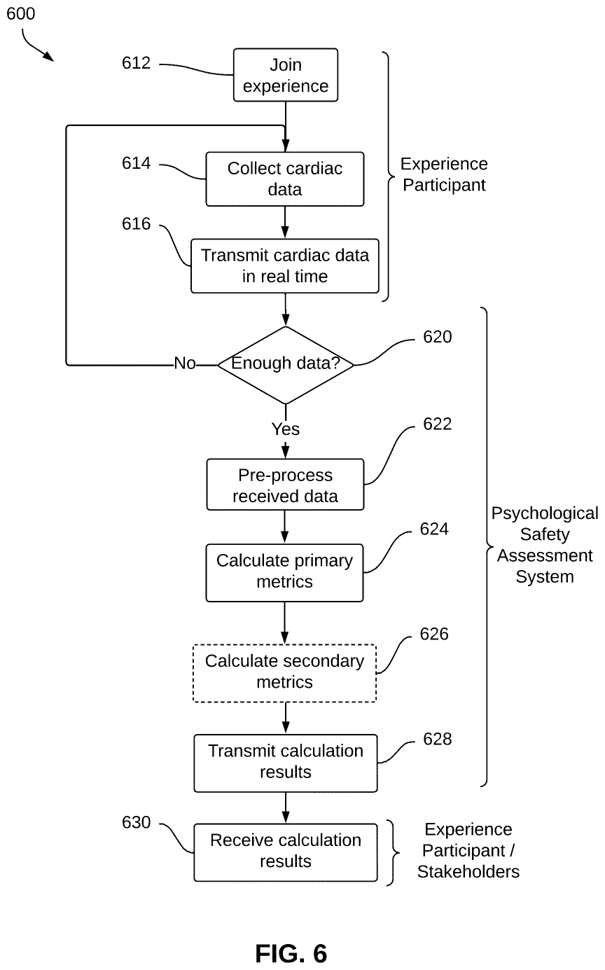

FIG. 6 shows a flow diagram for using a system for assessing the psychological safety level of an experience participant with presented content, in accordance with an embodiment. As shown in FIG. 6, in conjunction with FIG. 1, a process 600 begins when a participant (e.g., experience participant 110) opts to join an experience to be assessed (e.g., a live event or media content 170) in a step 612. Step 612 may include, for example, an opt-in agreement through an application interface or a manual signing of a waiver, in which the experience participant agrees to share physiological data (e.g., heart rhythm data) and in some cases demographic information collected during the experience. Then, in a step 614, cardiac data is collected from the participant from a device that is transmitting at least once every 5 seconds, and the collected information is transmitted to the psychological safety assessment system (e.g., psychological assessment system 100). As shown in FIG. 6, steps 612, 614, and 616 are performed at devices controlled by the participant. It is noted that multiple participants may be performing steps 612, 614, and 616 at the same time to provide input data to the neuroscience assessment system. Then the collected information from one or more experience participants are analyzed by a psychological safety assessment system in steps 620-628.

Continuing to refer to FIG. 6, the cardiac data is received at the psychological safety assessment system to determine whether sufficient data has been collected (e.g., data has been collected for a predetermined amount of time, at least two minutes) in a decision 620. If the answer to decision 620 is NO, then process 600 returns to step 614 to continue to collect cardiac data. If the answer to decision 620 is YES, then process 600 proceeds to a step 620 to pre-process the collected data. Step 622 may be performed, for example, by ingestion data hub 120. Step 622 pre-processing includes but is not limited to, identifying and correcting anomalies in the heart rhythm (e.g., out of biological ranges), and finding data gaps and inserting missing values based on expected heart rhythm patterns. Then primary metric, psychological safety, is calculated in a step 624. Step 624 may include the psychological safety analysis steps as discussed above and be performed, for example, by neuroscience processing unit 130 by correlating the input data from participants with known neurophysiologic indicators, as described above. Psychological safety calculations occur for experiences that are of sufficient length and with enough prepossessed data to identify heart rhythm patterns (e.g., 2 minutes or greater). The analysis takes a “bin” approach where the experience is broken up into predetermined bins by the system of equal duration. In real time, once enough pre-processed data is captured to complete a bin, psychological safety is derived and ready for display. Optionally, secondary metrics may be calculated in a step 626 by, for example, behavior analysis unit 140 of FIG. 1. Such secondary metrics may include aggregated analyses of primary metrics from multiple participants, analysis of the primary metrics over the duration (or smaller intervals within the duration) of the experience, and predictions of anticipated participant behavior following the experience. The calculation results of the primary and/or secondary metrics are then transmitted to participants or stakeholders in a step 628, such as via application interface 114 and/or user interface aspects of workflow management unit 150 of the psychological safety assessment system 100. Finally, the calculation received by participants or other stakeholders (e.g., partner companies 160, content creators 162, or data aggregator in cloud server 164) in a step 630.

Commonly-used PPG data collection wearables such as smartwatches and fitness trackers, or using PPG approaches using a built-in camera of a smart device, such as fingertip contact photoplethysmography (e.g., measuring finger pulse by contacting a fingertip to a built-in camera of a smart device) or non-contact photoplethysmography (e.g., using the built-in camera of a smart device to measure heart rhythm data). Heart rhythm measurement may be performed by approaches other than PPG, as long as the heart rhythm data can be collected with sufficient accuracy and resolution to enable performance of the analytic processes described herein.

While the above disclosure describes various ways of performing neuroscientific analysis on individuals and groups in reaction to specific experiences, it is herein recognized that further insights may be gained by obtaining and analyzing neurophysiologic responses over longer term and within context, i.e., over space/location and time. Capturing, and analyzing neurophysiologic responses over longer time periods (that is, beyond a discrete experience such as an advertisement, movie clip, or concert) and within context is possible with the immersion and psychological safety assessment system described herein, which takes advantage of the proliferation of mobile PPG technology such as but not limited to PPG-enabled smartwatches, fitness wearables, and mobile devices such as mobile phones, tablets and the like. Further, collection of neurophysiological data in real time (i.e., within context, over time, and outside of the laboratory setting) as enabled by the mobile PPG technology in combination with the contextual assessment system described herein, allows the contextualized analysis as previously unavailable in the industry of neuroscience.

It is recognized herein that, by taking into consideration the context of neurophysiological measurements, it is possible to glean information about a particular individual or even a group of individuals. While previous applications of similar neurophysiological analysis systems, such as the immersion and psychological safety assessment systems described above, were generally concerned with the assessment of those parameters in multiple people participating in a particular experience, the present disclosure recognizes that the focal point of the assessment may be the neurophysiological health of an individual across a variety of experiences over time and context. For instance, the heart rhythms of a particular, participating individual in the neurophysiological assessment may be analyzed within the context of that participating individual's life occurrences, such as planned events on their calendar, sleep patterns, location, proximity of known users (e.g., friends and family) or crowds, traffic and/or weather conditions, and other contextual information. Such contextualization of the neuroscientific analysis may be used to quantitatively assess the participating individual's emotional wellbeing and other health conditions.

For example, a key application for the innovation described herein is the neurophysiological measurement and assessment of social engagement, and thereby social withdrawal and isolation. It is recognized in the literature that social withdrawal and isolation is a prodrome for a large set of disorders, including depression, anxiety, Parkinson's disease, several autoimmune disorders, heart failure, dementia, addictive disorders, among others (see, for example, 1) S. Grav, et al., “Association between social support and depression in the general population: the HUNT study, a cross-sectional survey,” Journal of Clinical Nursing, 21 (1-2), 111-120 (2012); 2) Rohde, N., D'Ambrosio, C., Tang, K. K. et al. “Estimating the Mental Health Effects of Social Isolation.” Applied Research Quality Life, 11, 853-869 (2016).; 3) National Academies of Sciences, Engineering, and Medicine, “Social Isolation and Loneliness in Older Adults: Opportunities for the Health Care System,” Washington, DC: The National Academies Press (2020); 4) Centers for Disease Control and Prevention. Loneliness and social isolation linked to serious health conditions; 5) N. K. Valtorta, et al., “Loneliness and social isolation as risk factors for coronary heart disease and stroke: systematic review and meta-analysis of longitudinal observational studies,” Heart, 102 (13), 1009-1016 (2016); 6) M. Prenger, et al., “Social symptoms of Parkinson's disease,” Parkinson's Disease (2020); and 7) B. Littorin, et al., “Family characteristics and life events before the onset of autoimmune type 1 diabetes in young adults: a nationwide study,” Diabetes Care, 24 (6), 1033-1037 (2001)). As such, monitoring for and identifying neurophysiological indications of social withdrawal may be crucial in the early recognition of these and other potentially life-threatening conditions.

For instance, as described above, immersion tracks peak emotional moments when the individual has positive social experiences and performs activities that are personally rewarding. Similarly, psychological safety may be measured as a neurologic metric contributing to emotional wellness. When people feel anxious or unsafe, they are unable to connect to others and engage in activities that produce immersion. Without psychological safety, they may feel stressed, irritable, and disconnected. Immersion and psychological safety may be accurately assessed in individuals using neurophysiological assessment tools by applying proprietary algorithms to PPG data. Additionally, it is recognized herein that emotional well-being is strongly correlated with the co-occurrence of the neurologic states of immersion and psychological safety, thus levels of emotional well-being may be assessed and predicted based on the assessment of immersion and psychological safety within context, such as over time, health conditions, and/or other factors in the life of the individuals being evaluated.

Further, additional aspects may be factored into the analysis methods described above, including the analytical consideration of the context in which the experiences take place. For example, the so-called “Five Ws and One H,” namely who, what, when, where, why, and how, can play a significant role in an individual's neurologic response to a particular experience. In particular, the “when” (i.e., the specific time, duration, and chronological context of the experience) and the “where” (i.e., the particular location, geographical context (e.g., a busy coffee shop, a crowded protest rally, a quiet museum), proximity to home, and other location factors) may be useful in contextualizing the neurologic response measured in an individual. The above described embodiments of neuroscientific analysis may be further refined by considering a variety of contextual parameters, which may also be obtained using the same algorithms applied to PPG data collection or through user interface queries through the wearable or a mobile application, for example. Additional data (such as “what”—i.e., the type of event or experience in which the individual is participating, and “where”—i.e., at what point in space is the individual at any particular point in time) that may be collected and integrated into the analysis include, and are not limited to:
1. Number and identity of nearby users (e.g., proximity of known users such as friends and family, and/or aggregated number of nearby users as correlated with the capacity of the specific venue);
2. Time (e.g., obtained via the timestamp of the wearable or mobile devices or other meta data);
3. Location (e.g., obtained via using a global positioning system (GPS) functionality, or Bluetooth beacons within a fixed spatial location);
4. Activity (e.g., obtained via calendar applications);
5. Weather conditions (e.g., obtained via commercial services for the known location);
6. Traffic conditions (e.g., obtained via commercial services for the known location);
7. Repetition (i.e., the number of times the individual has participated in the same experience over time, at the same location, with the same set of nearby users, relative to past experiences at the same location, etc.);
8. Demographics and health conditions of the individual (e.g., self-reported or clinically diagnosed “wellness,” experiencing a cough, sore throat, headache or other malady including mood states);
9. Usage of applications on the wearable or a mobile device (e.g., usage of social media applications, news applications, web browsers); and
10. Self-reported data, such as responses to a reminder prompt, survey results, and other methods of collecting information directly from the individual via software application, oral interviews, or written response.

Other contextual data may also be obtained, such as through wearable or mobile device(s) being monitored by the neuroscientific analysis system, and/or commercial services such as geolocation functionalities (e.g., Bluetooth beacon devices) as well as weather and traffic apps. In embodiments, aggregated data (e.g., from internet service providers, smart home system providers, cellphone service providers) may also be integrated into the contextual analysis. By taking into consideration such contextual data, the neuroscientific analysis may be refined in context, such as tracking changes over time, correlated with real time conditions, and in other previously unavailable ways. Some potential uses include, and are not limited to:
1. Tracking a particular individual's neurologic emotional well-being by monitoring immersion and psychological safety assessments as measured over time and in a more objective, rather than subjective, manner;
2. Assessing the location and/or time dependence of a community's well-being;
3. Deriving an understanding of the success of a particular event, including wellness or resiliency interventions, according to immersion assessments of the attendees as correlated with time, geospatial location, proximity of others, weather conditions, traffic conditions, and other contextual information; and
4. Analyzing the affinity of events to groups of people with various levels of immersion, psychological safety, and other metrics derived from wearable and mobile devices.

For example, the levels of immersion and psychological safety may be combined in analysis to quantify levels of emotional well-being. Such quantified results may then be compared with self-reported and third party observed/diagnosed levels of mood and/or energy to evaluate meaningful correlation between the neurophysiologic metrics and a self-reported, diagnosed or observed emotional and physical well-being. Further, by taking into account other contextual information, also collectable using the same devices used for collection of neurophysiologic data, further refinement of the analysis may be possible. A recently published study describes the use of immersion and psychological safety analysis data to predict mood and energy in the elderly (See Merritt, S. H., Krouse, M., Alogaily, R. S., & Zak, J. P., “Continuous Neurophysiologic Data Accurately Predict Mood and Energy in the Elderly,” Brain Sciences, 12 (9), 1240, 2022, https://doi.org/10.3390/brainsci12091240). Such heretofore unavailable systems for continuously assessing emotional well-being may provide an invaluable tool for early detection of mental and physical health challenges and the determination of when wellness and/or medical intervention(s) may be required.

In other words, by combining the neuroscientific measures described above with a variety of contextual information, both obtainable through the wearable and mobile devices and through external services, heretofore unavailable insights may be obtained over time for individuals and amongst groups of individuals. The neurophysiologic measures may also be provided to each user and/or stakeholders in real time. While the previously described collection and analysis of biometric data such as PPG measurements enables the performance of neuroscience analyses outside of the laboratory context, the embodiments described herein further enable these analysis within context, including trends over time, location, and social settings.

To implement such an assessment system enabling “neuroscience anytime, everywhere,” a neuroscientific analysis system includes, in certain embodiments, components that are capable of the following activities:
1. Gather real-time PPG data from one or more individuals engaging in either synchronous or asynchronous activities;
2. Ingest data across time and across individuals at sufficient data fidelity (e.g., 1 Hz, corresponding to second-by-second data collection);
3. Collect contextual data relevant to the desired analysis; and
4. Perform the desired analysis translating PPG data into neurophysiologic metrics (see, for example, the above-referenced related applications U.S. patent application Ser. No. 17/874,114 and U.S. patent application Ser. No. 17/974,609).

Optionally, the assessment system may further include additional features such as:
5. Communicate with the individuals and/or stakeholders regarding the analysis results, in real time or in a subsequent report;
6. Flag individuals in a cohort of individuals who deviate from their historical data or a cohort in a predetermined way (e.g., send an alert to the individual and/or other contacts if the analysis results indicate low emotional fitness);
7. Aggregate (and optionally anonymize) analysis results across multiple individuals;
8. Track analysis results in real-time and over time, optionally within context (e.g., by location, across multiple individuals); and
9. Display analysis results in a “neuro mapping” of the data across time, location, etc. (e.g., a neuroscience data “heat map” showing intensity of immersion/psychological safety/emotional well-being levels at a particular location for a fixed time interval, such as immersion levels across an amusement park over the month of July).

The real-time PPG data may be collected, for example, using a PPG-enabled sensor in a wearable device, large area monitoring devices (e.g., imaging devices such as thermal cameras capable of remotely capturing pulse information), and other suitable devices. Location tracking of the individual being analyzed may be performed directly via a global positioning system (GPS) integrated into a mobile device or a wearable device, or external hardware, for instance a Bluetooth-enabled beacon.

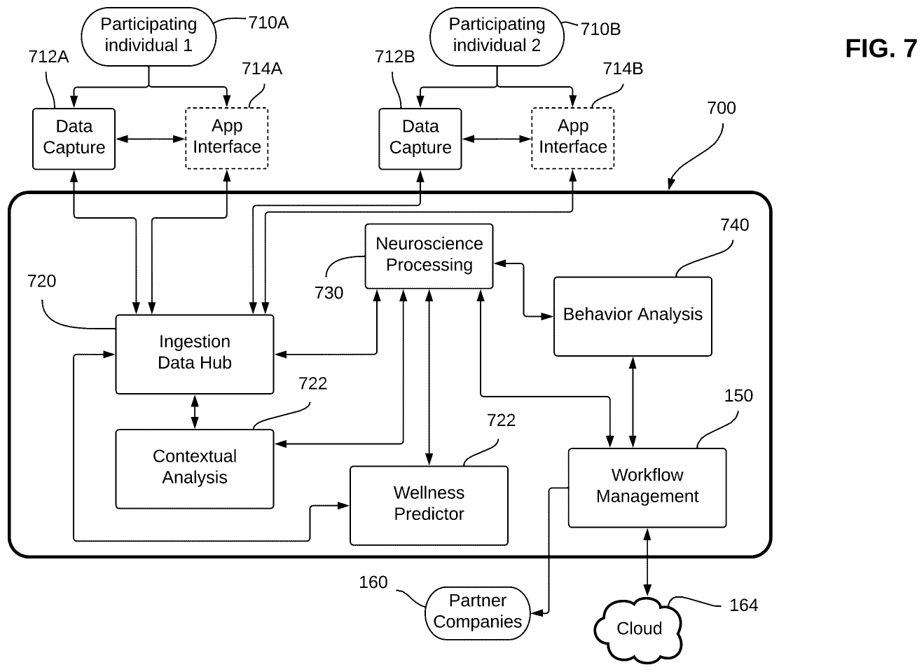

An exemplary system for assessing and/or predicting emotional well-being is illustrated in FIG. 7. As shown in FIG. 7, an assessment system 700 includes similar components as system 100 of FIG. 1 and is configured for capturing data provided by one or more participating individuals 710A and 710B. While only two participating individuals (also referred to elsewhere in this description as subjects or experience participants) are shown in FIG. 7, data may be simultaneously collected from more participating individuals, with the number of simultaneous individuals being limited by the data communication and processing bandwidth of assessment system 700.

Data capture mechanisms 712A and 712B, such as a mobile device or wearable device, collect PPG and additional contextual data from participating individuals 710A and 710B. Optionally, participating individuals 710A and 710B may be presented with an application interface or another user interface 714A and 714B to interact with assessment system 700. For instance, the application interface may pop up a daily reminder to the participating individual to fill out a health survey or a qualitative indication of their mood and/or energy the previous day. As another example, the participating individual may receive a daily email or text message request to report their health and wellness currently or in the recent past.

An ingestion data hub 720 is configured for receiving the PPG and contextual data from the participating individuals and processing the data so received. For example, in addition to the functionality of ingestion data hub 120 of FIG. 1, ingestion data hub 720 may perform functions such as identifying meta data associated with the contextual data, georeferencing the PPG data with location data, averaging PPG data over prescribed timeframes (e.g., hourly or daily) or variable timeframes (e.g., events retrieved from a smartphone calendar), and other pre-processing tasks.

Ingestion data hub 720 may then transfer the pre-processed data with a contextual analysis unit 722 for performing various types of contextual analysis as described above. For instance, contextual analysis unit 722 may group data from multiple participating individuals located within specific geographic boundaries in a given time frame to assess their immersion or emotional well-being. Such contextual analysis may add depth and provide further insights in comparison to stand-alone neuroscientific analysis without context.

Data from ingestion data hub 720 and contextual analysis unit 722 may be provided to neuroscience processing unit 730 for performing, for example, immersion, psychological safety, and/or emotional well-being quantification as described above. In particular, neuroscience processing unit 730 is configured to combine the pre-processed PPG data from ingestion data hub 720 with context information from contextual analysis unit 722 for performing contextualized neuroscience analysis. In certain embodiments, neuroscience processing unit 730 may include a processing engine trained by machine learning to use the contextualized neuroscience analysis to classify the analysis results into, for instance, different levels of emotional well-being. As an example, the neuroscience processing unit may use machine learning to further process the contextualized data to classify whether a particular participant (or a group of participating individuals) may be classified as having low mood or low energy, based on the received PPG and context data. A behavior analysis unit 740 may further consider the results of the neuroscience processing analysis as well as additional operations such as aggregate profiling, vertical analysis, and pattern analysis.

Optionally, neuroscience processing unit 730 may be further connected with a wellness predictor unit 742, which may extrapolate predictive data from the contextualized neuroscience analysis results of neuroscience processing unit 730. The emotional wellness predictor unit 722 may include, for example, a processing engine trained by machine learning to predict future values of, for example, mood or energy levels based on the analysis results from neuroscience processing unit 730.

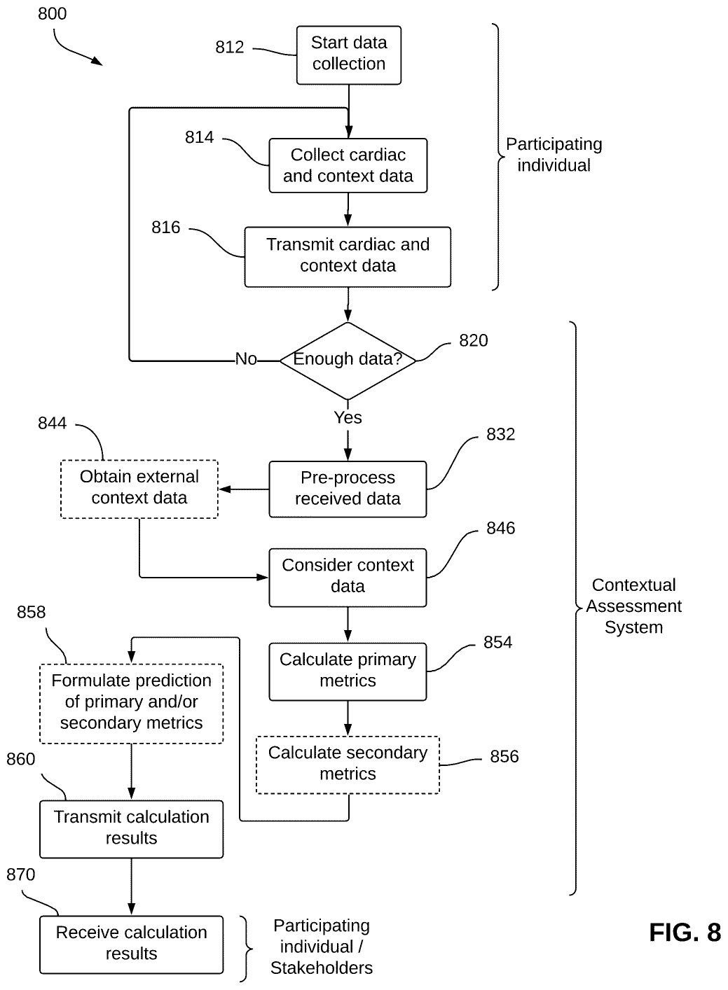

FIG. 8 shows a flow diagram for using a system for assessing and/or predicting emotional well-being of one or more individuals, in accordance with an embodiment. As shown in FIG. 8, a process 800 begins with activities related to a participating individual. A step 812 to start the data collection is initiated when the participating individual starts data collection related to a specific activity (e.g., a concert or a movie) or, in this case of neuroscientific assessment integrated with contextual analysis, consents to collection of neurophysiologic (e.g., cardiac) measurements while participating in normal daily activities. Such data including cardiac and context information may be collected in a step 814, and the data including cardiac and context information may be transmitted to an assessment system (e.g., assessment system 700 of FIG. 7) in a step 816.

In embodiments, the transmission of the data may take place in real time via, for example, Bluetooth, wireless, wired, cellular, or other types of transmission mechanisms. Alternatively, the data may be stored at the collection device and transmitted at specific times, such as hourly or daily.

It is noted that joining step 812, collection step 814, and transmission step 816 may simultaneously be performed by multiple participating individuals, if the assessment system is configured for simultaneously receiving and processing data from multiple individuals. In fact, collection of data from multiple participants within a given time frame and/or a location may provide valuable contextual information that can be used in refining the neuroscientific analysis described herein.

Continuing to refer to FIG. 8, subsequent steps may be performed by a contextual assessment system, such as assessment system 700 of FIG. 7. A determination is made whether enough data has been collected in a decision 820. If the answer to decision 820 is NO, then process 800 returns to step 814 to collect additional data. If the answer to decision 820 is YES, the process 800 proceeds to a step 832 to pre-process the received data. For example, pre-processing step 832 may be performed by ingestion hub 720 of FIG. 7.

Optionally, process 800 includes a step 844 to obtain external contextual data, such as weather, traffic, social media, self-reporting, and other context data as described above from external sources, such as public databases. Such external contextual data may be considered along with the context data collected from the data collection device used by the participating individual(s).

In a step 846, the contextual data is considered, for example, in contextual analysis unit 722 of FIG. 7. The consideration may include, for example, grouping of participating individuals located within close proximity of each other at a given time, the tracking of the collected data over time for a given individual, cross referencing of the collected PPG data with traffic information from a public database, and other activities. The contextualized PPG data may then be used to calculate primary metrics, such as immersion, in a step 854. Optionally, secondary metrics, such as comparison of the primary metrics over time or to aggregated primary metrics, calculating predicted primary and/or secondary metrics values at a future time point, such as predicting the mood and/or energy level of a participating individual, given particular levels of a primary metric, and others, may be calculated in a step 856.

Further, in an optional step 858, the primary and secondary metrics calculated may be further considered with the context data in order to formulate a prediction of the primary and/or secondary metrics. For instance, based on the processed cardiac and context data over time, prediction(s) may be made regarding a primary metric (e.g., immersion) or a secondary metric (e.g., psychological safety, energy, well-being). In an example, given the collected cardiac data for a given individual over time, a prediction may be made regarding the given individual's energy level or sense of well-being at a future time, such as the likelihood of the given individual being emotionally resilient in the near future given trends in the collected cardiac data over time. The calculation results are then transmitted by the contextual assessment system to the participating individual(s) and other stakeholders, such as caregivers or medical professionals, in a step 860. The participating individual(s) and/or stakeholders receive the calculation results in a step 870, such as displayed in a summary format in a user interface of a software application.

The present disclosure provides a way to measure neurophysiological data of one or more individuals over time and within the context of real life through the use of mobile PPG collection technology, such as smart watches and fitness sensors. In this way, the data collected allows for neuroscientific assessment for longer, uninterrupted periods of time (e.g., 24 hours a day), across as long a period of time as desired by the individual (e.g., days, weeks, months years).

Further, the embodiments described above enable analysis of the neurophysiological data within context (i.e., the 5Ws and H). For instance, the ability to process measurements over a longer time period, including provisions to manage and parse the quantity and variability in the collected data, enables assessment over many experiences and locations for one or more individuals, rather than at a discrete event (e.g., a lab experiment) as is the current practice.

Importantly, prior research has shown that prior self-reported mood (e.g. day before) was a poor predictor of current mood, indicating that self-reported data alone are unlikely to have sufficient predictive value (see, for example, van Breda, et al., “Exploring and comparing machine learning approaches for predicting mood over time,” Innovation in Medicine and Healthcare 2016, vol. 4, pp. 37-47). In contrast, by combining neurophysiological data capture over time and, optionally, within the context of other parameters, assessment system 700 enables assessment and prediction of mood and energy in one or more users, providing a clinically-useful indicator of mood variation in context.

The continuity of measurement, across time and locations, enables performance of neuroscience assessments within context. Further contexts may include, for example, geolocation and integration with other applications on the individual's mobile device (e.g., calendar, proximity of other users, social media information, camera, health tracking, and others). Further, the neuroscientific assessment and contextual data may be used to assess the individual's health in general or for specific conditions, such as emotional well-being.

### Example: Continuous Neurophysiologic Data Monitoring to Predict Mood and Energy in the Elderly

As an exemplary embodiment, the neurological assessment system with contextual data may be implemented to predict the mood and energy in a particular population, such as a group of elderly individuals in a retirement community. The approach described above to combine contextual information with analysis of PPG data has been used to successfully predict low mood and low energy in the assessed individuals.

Depression is a primary public health concern globally. The elderly are particularly vulnerable to depression due to age-related neural atrophy, hypertension, and social isolation. For example, many elderly persons are at an elevated risk of clinical depression because of isolation from family and friends and a reticence to report their emotional states.

Further, chronic low mood increases morbidity and mortality, especially in older adults. When people experience low moods that last for two weeks or more, they are diagnosed as clinically depressed. The lifetime incidence of depression is 14.6% for adults in developed countries, and women are approximately twice as likely as men to have an episode of depression. In the U.S., those aged 65 and older have a one in four depression risk. Life events can increase the likelihood of depression in seniors, including declining health, financial straits, losses of loved ones, reduced social interactions, inadequate healthcare, and the inability to participate in activities. Depression in old age is also a risk factor for dementia.

On the other hand, positive affect has a host of favorable impacts on health in the elderly, including a lower risk of cardiovascular disease, a reduction in pain, increased exercise, improved immune function, and better social relationships. It is likely that the causal flow connecting positive mood to improved health is bidirectional and depends in part on one's genetics. The importance of mood states on healthspan demands a more fundamental understanding of the causes of mood variations.

There are a variety of ways to inhibit the onset of depression, including social support, psychological counseling and pharmacotherapy. Such interventions are more effective if a decline in mood can be identified before a major depressive episode occurs. The ability to passively assess mood states using technology, such as using the continuous monitoring with contextual analysis approach described herein, would be an important public health advance. Not only is there a great need to predict moods, but the use of neural data obviates the need to constantly query individuals. Self-reports tend to be inaccurate, especially in the elderly. Colloquially, people may be “worried to death,” and indeed, there is an extensive literature relating negative mood states and clinical depression to anxiety.

Depressive symptoms in seniors may arise when individuals no longer enjoy activities (anhedonia). However, even with observation, it may take weeks or months to correctly classify an individual as depressed since variations in moods are common. When depressive symptoms are identified early, the prognosis for patients is substantially improved. The interaction between the quality of social activities and mood has the potential to be measured neurophysiologically.

Data that quantify activities and physiology using wearable technologies has exploded, and several approaches to predicting low moods have been investigated in the past. For example, using smartwatches and machine learning to analyze sleep as depressive episodes are associated with disordered sleep patterns. Low mood and depression have been predicted by applying artificial intelligence techniques to digital traces. A typical approach employs natural language processing for social media posts and chats. Other research has used machine learning to predict moods using song choices, street views, pictures of faces, and images from video conferences. These approaches are convenient because they use publicly available data without the need for direct measurement of participants. However, a shortcoming of this approach is the use of surveys of nonparticipants to assess the moods of participants. This issue induces bias in the dependent variables as it is known that people inconsistently identify others' moods. As a result, the predictive accuracy of these models seldom exceeds the 70-80% range.

Yet the availability of these new data may make it possible to predict mood states, and thereby well-being, using physiology in order to create interventions to reduce or eliminate the degradation of health from persistent negative affect, by combining the analysis of data gathered over time with contextual information, as recognized herein. As an example, the present exemplary embodiment enables the establishment of a relationship between self-reported moods to neurophysiologic data collected directly from study participants. Such assessments are known to be a difficult task as consciously filtered self-report measures in and of themselves are typically unrelated to neural activity.

As recognized herein, a first step in creating a potential early detection measure for melancholia is to determine if neurophysiologic measures are associated with mood. The implementation described herein used a sample of healthy seniors, rather than a clinical population, in order to establish a combination of neural measures derived from a wearable sensor would predict changes in mood states.

In the present exemplary embodiment, neurophysiologic data were collected continuously from 24 participating individuals for three weeks at 1 Hz and averaged into hourly and daily measures, while mood and energy were captured with self-reports. Two neurophysiologic measures averaged over a day predicted low mood and low energy with 68% and 75% accuracy. Principal components analysis showed that neurologic variables were statistically associated with mood and energy two days in advance. Applying machine learning to hourly data classified low mood and low energy with 99% and 98% accuracy. Two-day lagged hourly neurophysiologic data predicted low mood and low energy with 98% and 96% accuracy. In this way, continuous measurement of neurophysiologic variables may be used as an effective way to reduce the incidence of mood disorders in vulnerable people by identifying when interventions are needed. Specific details of the experimental embodiment are described, for example, in Merritt, et al., “Continuous Neurophysiologic Data Accurately Predict Mood and Energy in the Elderly,” Brain Sciences, vol. 12, 1240 (2022), which publication is incorporated herein in its entirety by reference.

Particularly, participating individuals were sent an email every day at 6 a.m. and asked to complete an online survey reporting their mood, health, and energy from the day before. If no response was collected by noon, participants were reminded via email and text to complete the survey. Missing data from the self-reports was less than 4%. Retrospective self-reports were used to capture perceptions of the previous day's mood because contemporaneous self-reports may induce a bias towards one's acute mood state, but can reduce accuracy due to poor recall and misattribution of arousal. This approach decreases the likelihood of significant associations to physiologic signals associated with retrospective mood reports. In order to reduce the burden of data collection, which required wearing a smartwatch daily and charging it overnight, the only additional information obtained from residents was their biological sex and whether they were ill.

An assessment system (e.g., assessment system 700 of FIG. 7) was used to measure neurophysiologic responses collected at 1 Hz. The independent variables obtained from the assessment system were average immersion for each day and average psychological safety. As described above, neurologic immersion combines signals associated with attention and emotional resonance and measures the value the brain places on social experiences. The attentional response is associated with dopamine binding to the prefrontal cortex, while emotional resonance is related to oxytocin release from the brainstem.

In addition, we defined a variable called peak immersion (PI) by the following equation:

$$PI = {\int }_{t = 0}^{T} ( {v}_{it} > {M}_{i} ) {d}_{t} / {Im}_{i} \qquad ( Eq . 1 )$$

where vᵢₜ is the hourly average neurophysiologic immersion for each participant in day i at time t to the end of the day at time T, Mᵢ is the median of the average hourly time series of immersion for day i plus the standard deviation of the hourly data for day i for each participant, and this value is divided by the sum of total immersion Imᵢ for each person for each day i. That is, peak immersion PI cumulates the highest immersion moments for an individual during the day capturing high-value social experiences relative to the immersion from total social experiences.

Average hourly immersion and psychological safety were used to build machine learning (ML) models to assess predictive accuracy compared to models using daily data. The ML models included regularized logistic regressions, random forests (RF), and support vector machines (SVM).

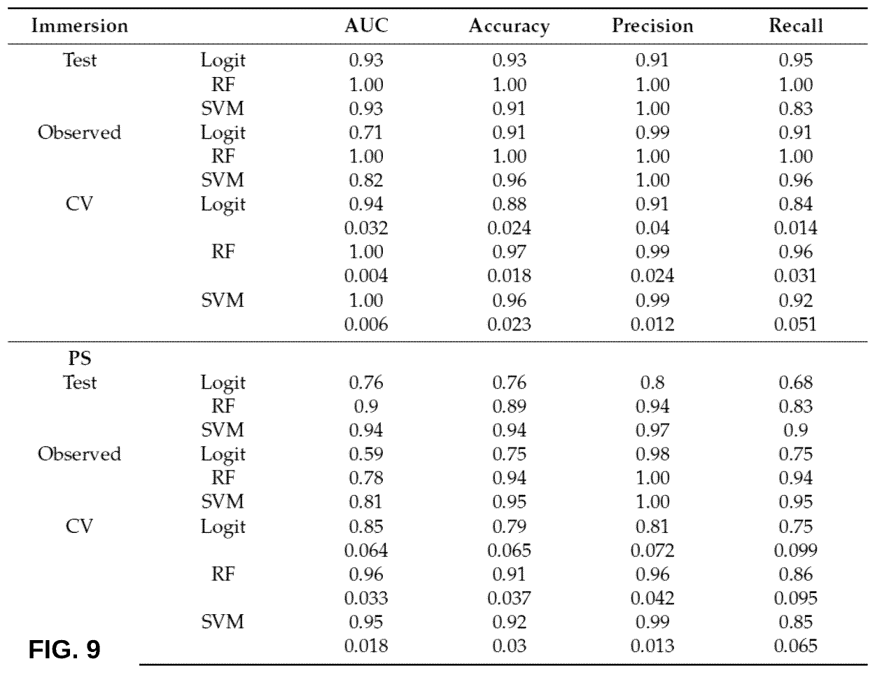

The assessment of immersion, psychological safety, and peak immersion data revealed that only PI was statistically related to Mood. Logistic regression estimates to examine when a participant was likely to have low Mood. The ML estimations classifying Mood fit the data well in the test set (AUCs>0.90), as summarized in FIG. 9. In particular, ML classification of Mood using Immersion and PS in regularized logit, random forest (RF), and support vector machine (SVM) estimations, and CV is cross-validation were performed. All models maintained high scores across the five folds indicating they were not overfit. Predictive accuracy for the ML models using Immersion was very high for the observed data; correct classification of Mood ranged from 99 to 100%. Using PS in the ML estimation was nearly as accurate, ranging from 98 to 100%.

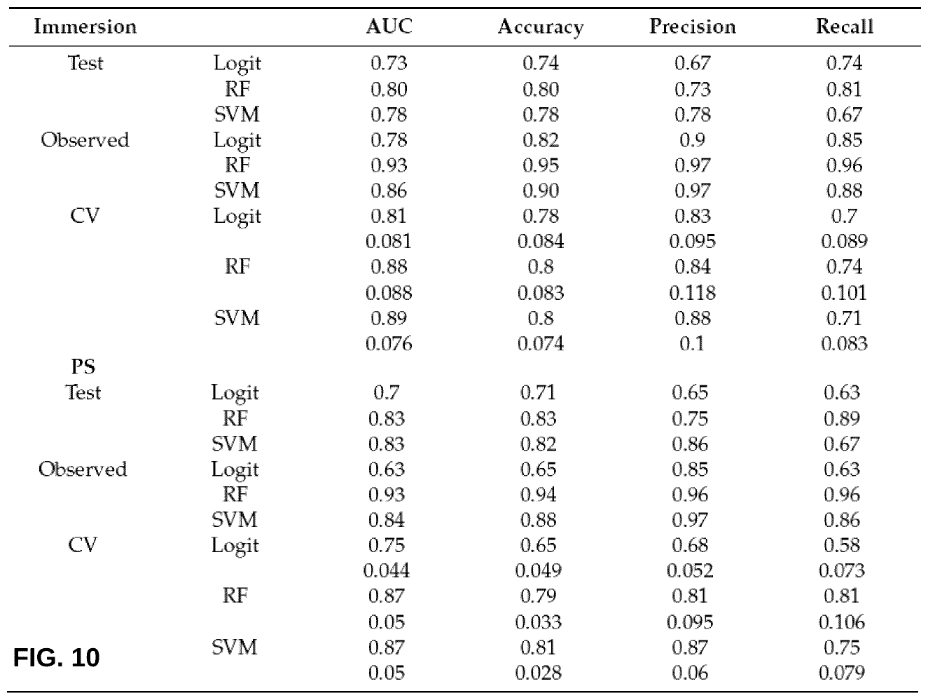

Repeating the ML estimations using Energy as the dependent variable, 27 features from the Immersion data and 11 from PS were extracted using logit (C=100, penalty=“I2”), RF (max_features=‘sqrt’, ‘min_samples_leaf’=2, ‘min_samples_split’=[2, 5]), and SVM (C=10, kernel=‘rbf’). The ML estimation results are summarized in FIG. 10. Particularly, FIG. 21 shows ML classification of Energy using Immersion and PS in regularized logit, random forest (RF), and support vector machine (SVM) estimations, where CV is cross-validation.

The predictive accuracy of Mood with neural data two days prior to the self-report was also performed in order to extend the principal component findings. The two-day lagged goodness of fit mirrored the contemporaneous estimations. Further, immersion and psychological safety may be used to predict Energy from the two-day lagged data.

Predictive accuracy continued to be quite high using hourly data lagged two days. Immersion, in particular, strongly predicted Mood using the lagged data, and two-day lagged ML models were nearly identical in their accuracy compared to the contemporaneous data estimates.

As demonstrated by the present example, high frequency neurophysiologic measures may be used to accurately predict self-reported emotional states. Passive and continuous data collection, as used here, appears to be an effective way to monitor emotional wellness. The exemplary analysis shows that declines in emotional states can be predicted two days in advance with high accuracy. Such data make it possible for family members or clinicians to check in on the elderly in order to halt a decline before a potential mental health crisis occurs. Further, the present exemplary study shows that neural measures can be used to monitor the quality of life. Neural predictors of emotional states can also be used to identify the physiological processes inhibiting satisfaction with one's life so that interventions are focused and effective.

The results are surprising because 1 Hz neurophysiologic data were averaged into hourly and daily measures that we expected would return to a long-term equilibrium. That is, daily observations were statistically independent of each other, yet had predictive value as a group. Estimations using daily data predicted low Mood and low Energy with 68-75% accuracy using just three neurophysiologic variables in a standard logistic regression, controlling for illness and sex. These models were extremely accurate, correctly classifying the dependent variables with 84-100% accuracy.

Psychological safety on a daily basis does not appear to influence Mood or Energy. However, this variable accurately predicted both dependent variables when measured hourly. Further, the use of an assessment system such as described herein (e.g., assessment system 700 of FIG. 7) enabled full processing of neurophysiologic measures captured at scale.

In summary, contextual analysis can provide meaning to the neurophysiologic data collected, namely immersion and psychological safety scores. For example, the assessment system embodiments described above may be used for a variety of uses, such as:
Identify what, for instance, experiences, time of day, time of week, affects immersion and psychological safety. For instance, what repeated experiences or experience categories (e.g., work, leisure, family time) lead to more immersion or psychological safety. In providing this context, an individual identify what moments bring greater value to them, and potentially greater emotional well-being.
Measuring over time, as contextual information, can provide data to the neurophysiologic system to identify emotional wellbeing, energy, and mood for a future point in time (e.g., the following day).
The system can make recommendations for experiences that have led to higher immersion or psychological safety in the past. The system can make these recommendations when, for instance, the individual has had a sustained period of time where immersion or psychological safety values are low, or when the individual has not had
The system can share contextual and neurophysiological data with people the individual user decides to connect with, providing insight on users interpersonal relationships.

Further applications of the assessment systems and methods described herein are contemplated and are considered to be a part of the present disclosure.

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms-even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “protrusion” should be understood to encompass disclosure of the act of “protruding”—whether explicitly discussed or not—and, conversely, were there only disclosure of the act of “protruding,” such a disclosure should be understood to encompass disclosure of a “protrusion.” Such changes and alternative terms are to be understood to be explicitly included in the description.

1. A neurophysiologic assessment system for assessing primary metrics of a participant and extracting secondary metrics over time, the system comprising:
an ingestion data hub for receiving heart rhythm data collected from the participant and processing the heart rhythm data received to generate clean data;
a neuroscience processing unit for receiving and analyzing the clean data at predetermined intervals to generate primary metrics;
a contextual analysis unit for receiving and analyzing external contextual data;
a behavior analysis unit for receiving and analyzing the clean data, the primary metrics, and the external contextual data to generate secondary metrics; and
a workflow management unit for controlling the ingestion data hub, the neuroscience processing unit, the contextual analysis unit, and the behavior analysis unit.

2. The neurophysiologic assessment system of claim 1, wherein the secondary metrics include at least one of emotional wellbeing, energy, mood, and peak immersion value for a given time frame.

3. The neurophysiologic assessment system of claim 1, wherein the external contextual data include at least one of location, proximity of related persons, time of day, day of a week, weather conditions, traffic conditions, measured activity levels, calendar events, biofeedback, survey results and information provided by the participant, tracked usage of software applications, engagement with social media, and sleep patterns.

4. A method for assessing emotional well-being of an individual, the method comprising:
collecting data related to the individual, wherein the data include neurophysiologic measurements and contextual information;
transmitting the data to an assessment system;
pre-processing the data;
calculating primary metrics; and
calculating secondary metrics,
wherein collecting data includes collecting neurophysiologic measurements in at least 1 Hz intervals,
wherein calculating primary metrics includes using the neurophysiological measurements to calculate at least one of immersion and psychological safety, and
wherein calculating secondary metrics includes combining the primary metrics so calculated with contextual information to generate the secondary metrics.

5. The method of claim 4, wherein calculating secondary metrics further includes generating a predicted value of at least one primary metric and a secondary metric at a future time point.

6. The method of claim 4, wherein calculating secondary metrics includes calculating at least one of emotional wellbeing, energy, mood, and a peak immersion value for a given time frame.
